(19) United States
(12) Patent Application Publication
(10) Pub. No.: US 2024/0027466 A1
(43) Pub. Date: Jan. 25, 2024

(54) METHOD OF ISOLATING AND MEASURING DESMOSINE AND ISODESMOSINE IN ELASTIN

(71) Applicant: Nutraceuticals International Group LLC d/b/a Nutraceuticals Group, Little Falls, NJ (US)

(72) Inventor: David ROMEO, Washington Twp., NJ (US)

(21) Appl. No.: 18/330,397

(22) Filed: Jun. 7, 2023

Related U.S. Application Data
(60) Provisional application No. 63/369,070, filed on Jul. 22, 2022.

(51) Int. Cl. G01N 33/68; G01N 1/34; C07D 213/55

(52) U.S. Cl. CPC G01N 33/6812; G01N 1/34; C07D 213/55

(57) ABSTRACT

A method of isolating desmosine and isodesmosine in elastin includes hydrolyzing a sample of elastin and separating the desmosine and isodesmosine within a high-performance liquid chromatography (HPLC) column. A mobile phase gradient includes a first solvent solution of MSA (methane sulfonic acid) and HSA (sodium hexanesulfonate) and a second solvent solution of formic acid in Acetonitrile. The desmosine and isodesmosine may be measured as eluents based upon a HPLC chromatogram.

### PRIORITY APPLICATION(S)

This application is based upon provisional application Ser. No. 63/369,070 filed Jul. 22, 2022, the disclosure which is hereby incorporated by reference in its entirety.

### FIELD OF THE INVENTION

The present invention relates to the field of elastin, and more particularly, this invention relates to isolating and measuring the isomeric amino acids desmosine and isodesmosine in elastin.

### BACKGROUND OF THE INVENTION

Skin properties are characterized by interactions between cells, proteins and numerous cellular function. A complex network of interlaced collagen fibrils in the dermis provides support to the epidermis, and together with elastin and microfibrils, gives the skin its elasticity and resilience. Collagen occurs as a triple helix made from three chains of polypeptide proteins containing glycerine, hydroxy proline and proline amino acids, and forms the connective tissues in humans. It is particularly abundant to form parts of the skin and is found in tendons that connect muscle to muscle, and is part of the ligaments that join bones and muscle together.

Elastin, on the other hand, is a key extracellular matrix protein that is critical to the elasticity and resilience of many vertebrate tissues including large arteries, lung, ligament, tendon, skin and elastic cartilage. Loss of and changes to elastic fibers are major contributing factors to skin aging. Chronological aging, ultraviolet exposure and inflammation, all increase the release of elastases, which are the enzymes responsible for elastin degradation, particularly, the human neutrophil and macrophage produced elastases. These elastases degrade elastic fibers resulting in skin sagging and wrinkling.

Oral intake of elastin peptides has shown promising results in increasing the amount of collagen and elastin in aging skin and reducing skin conditions associated with chronological aging and photo-aging, such as skin roughness, stiffness, lack of elasticity, and increase of wrinkles. Some clinical and experimental evidence suggests that this mechanism is related to the upregulation of elastin synthesis-related factors, thus improving the production of elastin. For example, in one Japanese clinical study, elastin hydrolysate enhanced the proliferation of fibroblasts and elastin synthesis. The maximum proliferation response was observed at 25 ng mL(−1) Pro-Gly (prolyl-glycine). The ingestion of elastin hydrolysate improved skin conditions, such as increasing elasticity, reducing the number of wrinkles, and increasing blood flow. Elasticity improved by 4% in the elastin hydrolysate group compared with 2% in the placebo group. Thus, evidence shows that elastin hydrolysate activates human skin fibroblasts, which have beneficial effects on skin conditions. Elastin hydrolysate also prevents skin aging and improves skin health by increasing and maintaining its elasticity.

Elastin is a stable protein and has an extracellular matrix, which includes collagen, glycoproteins, glycosaminoglycans and proteoglycans. A unique aspect of elastin is its structure that includes the unique amino acids/proteins desmosine and isodesmosine, which are found only in elastin. Desmosine cross-links with its isomer, isodesmosine, thus giving elasticity.

There have been advancements in skin molecule technologies in recent years. For example, some products have been developed from Atlantic cod fish as a water soluble, additive free and hydrolyzed form of elastin. Because it is important to verify the quantitative levels of desmosine and isodesmosine, improvements to test and verify these protein/amino acid markers desmosine and isodesmosine would be beneficial in order to give a verifiable, validated and scientifically rigorous and accurate numerical value.

### SUMMARY OF THE INVENTION

This summary is provided to introduce a selection of concepts that are further described below in the Detailed Description. This summary is not intended to identify key or essential features of the claimed subject matter, nor is it intended to be used as an aid in limiting the scope of the claimed subject matter.

In general, a method of isolating desmosine and isodesmosine in elastin may comprise hydrolyzing a sample of elastin, and separating the desmosine and isodesmosine in the hydrolyzed sample of elastin within a high-performance liquid chromatography (HPLC) column, using a mobile phase gradient comprising a first solvent solution of MSA (methane sulfonic acid) and HSA (sodium hexanesulfonate) and a second solvent solution of formic acid in Acetonitrile.

The HPLC column may comprise a C18 HPLC column operating at about 30 to about 40 degrees Centigrade. The first solvent solution may comprise about 0.098 M to about 0.102 M MSA and about 5.88 mM to about 6.12 mM HSA at about pH 1.90 to about pH 2.1. The second solvent solution may comprise about 0.196% to about 0.204% formic acid in Acetonitrile at about 0-5% in about 4 to about 6 minutes, 5-12% in about 12-18 minutes, and 12-5% in about 12 to about 28 minutes.

The hydrolyzing may comprise washing the sample of elastin in chilled alcohol at less than about 10 degrees Centigrade. The washing may comprise mixing the sample of elastin with the chilled alcohol for about 20 to 40 seconds, and letting stand at about 30 to about 10 degrees Centigrade for about 5 to 15 minutes, followed by mixing for about 5 to 15 minutes at about 0 to 10 degrees Centigrade. The hydrolyzing the washed sample of elastin may include vacuum hydrolysis with HCl. The vacuum hydrolyzing the washed sample of elastin may be at about 100 to 120 degrees Centigrade for about 20 to 28 hours. The drying the hydrolyzed sample of elastin using nitrogen gas may be at about 50 to about 70 degrees Centigrade. The amount of desmosine and isodesmosine may be measured as eluents from the HPLC column at about 275 nanometers based upon a HPLC chromatogram.

### BRIEF DESCRIPTION OF THE DRAWINGS

Other objects, features and advantages of the present invention will become apparent from the Detailed Description of the invention which follows, when considered in light of the accompanying drawings in which:

FIG. 1 is a structural formula for the amino acid desmosine found uniquely in elastin.

FIG. 2 is a structural formula for the isomer isodesmosine that crosslinks with desmosine.

FIG. 3 is a high-level flowchart of a method of isolating desmosine and isodesmosine in elastin.

FIG. 4 is a high-level flowchart of a method of measuring desmosine and isodesmosine in elastin.

FIG. 5 is a graph HPLC chromatogram for a desmosine/isodesmosine reference.

FIG. 6 is an example chromatogram of an elastin hydrolysate sample using the method for measuring as in FIG. 4 in accordance with a non-limiting example.

### DETAILED DESCRIPTION

Different embodiments will now be described more fully hereinafter with reference to the accompanying drawings, in which preferred embodiments are shown. Many different forms can be set forth and described embodiments should not be construed as limited to the embodiments set forth herein. Rather, these embodiments are provided so that this disclosure will be thorough and complete, and will fully convey the scope to those skilled in the art.

The physiological characteristics of elastin are highly influenced by the presence of the amino acids desmosine and isodesmosine, which are unique to elastin. Desmosine and isodesmosine are positional isomers of a lysine conjugate having four lysines that combine to form a pyridinium group and are operative as cross-links exclusively in the elastin protein. Desmosine and isodesmosine serve as the cross-linking molecules binding the polymeric chains of amino acids into the three-dimensional network of elastin.

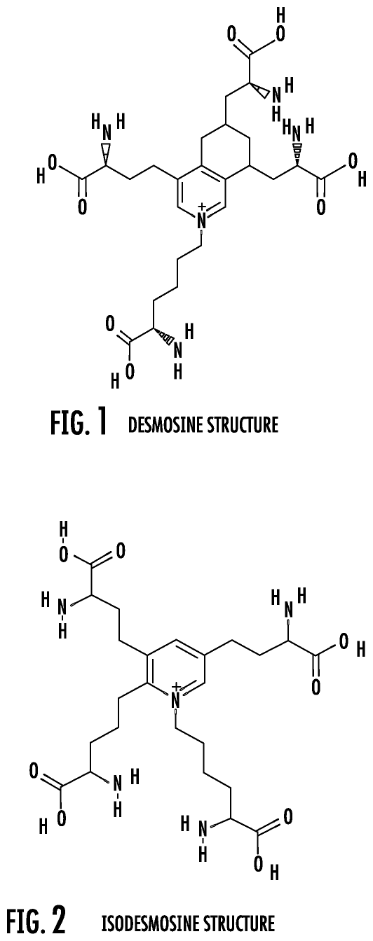

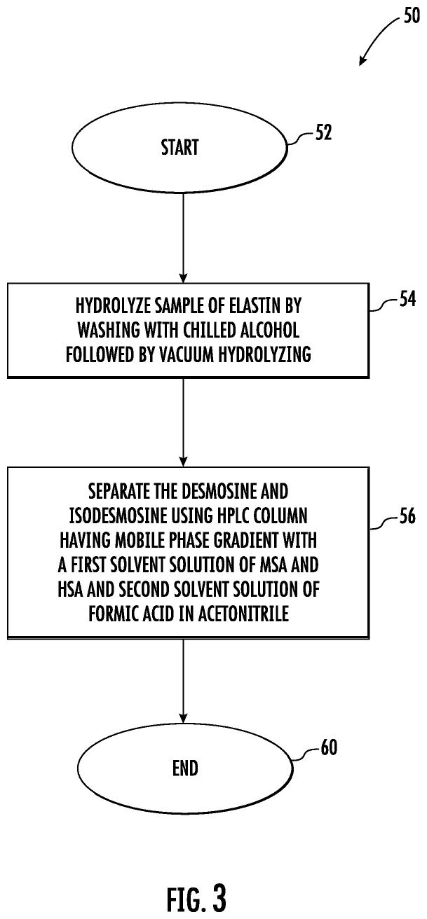

Reference is made to FIGS. 1 and 2 that illustrate respectively the desmosine chemical structure and the isodesmosine chemical structure. These unique amino acids form the cross-links for the elastin protein molecule that includes tropoelastin molecules, which include hydrophobic and hydrophilic components that contain many lysine amino acids along with the glycine and proline molecules. The elastin includes the tropoelastin molecules that are linked together with the lysine and include alanine, proline and glycine residues. Unlike in collagen, the glycine residue does not occur at each third position, and a helix is not formed as in collagen. Elastin has great elasticity and is considered to be a thousand times more elastic than collagen, which enables the lungs to recoil and blood vessels to expand and contract when required. Elastin also helps regulate cell behavior and binds with other proteins in the extracellular matrix of connective tissues. It provides a snap back property to cellular and body structures when they are stretched. Elastin production usually occurs in the early developmental stages in childhood.

As noted before, desmosine and its isomer isodesmosine both include four lysine residues, allowing for bonding the multiple peptide chains. The four lysine groups combine to form the pyridinium nucleus as shown in FIGS. 1 and 2, which may be reduced to neutralize associated positive charges and increase the hydrophobicity. The four lysines may form side chains around the pyridinium nucleus with exposed carboxyl groups. The difference between desmosine and isodesmosine includes an exchange of a lysine side chain on carbon 1 with a proton on carbon 5. Desmosine is associated with alanine, bonding with it on the N terminal side. Thus, this alanine association allows it to bond well with pairs of tropoelastin to form elastin and elastin networks.

It is challenging to differentiate desmosine and isodesmosine. There are some mass spectrometry techniques that help differentiate the two isomers and some high-performance liquid chromatography techniques. These techniques have not been found effective as a validated method to test and verify accurately these protein/amino acid markers in different skin conditioners that make use of elastins, such as elastin hydrolysate.

In accordance with a non-limiting example, a method for isolating desmosine and isodesmosine and measuring their quantity in a sample of elastin has been developed that is scientifically rigorous and accurate. The test method includes the chromatographic identification and measurement of the isomeric amino acids desmosine and isodesmosine from elastin protein hydrolysate. The method includes: (1) hydrolysis of elastin protein powder with 6N HCl; (2) drying of hydrolysate using N₂; (3) separation of desmosine and isodesmosine through ion pair chromatography, taking advantage of their chemical nature as a positively charged pyridinium ring and negatively charged alkyl sulfonate group; and (4) quantification at λ=275 nm.

Referring now to FIG. 3, there is illustrated a high-level flowchart of a method of isolating desmosine and isodesmosine in elastin indicated generally at 50. The process starts (Block 52) and a sample of elastin is hydrolyzed by washing the chilled alcohol followed by vacuum hydrolyzing (Block 54). The desmosine and isodesmosine are separated using a HPLC column having a mobile phase gradient with a first solvent solution of MSA (methane sulfonic acid) and HSA (sodium hexanesulfonate) and a second solvent solution of formic acid in Acetonitrile (Block 56). The process ends (Block 60).

The isolated desmosine and isodesmosine can be used in small quantities for research and development and analysis of the elastin hydrolysates. It is also possible to use the method to measure the desmosine and isodesmosine as the eluents from the HPLC column based upon an HPLC chromatogram and comparing with a reference standard.

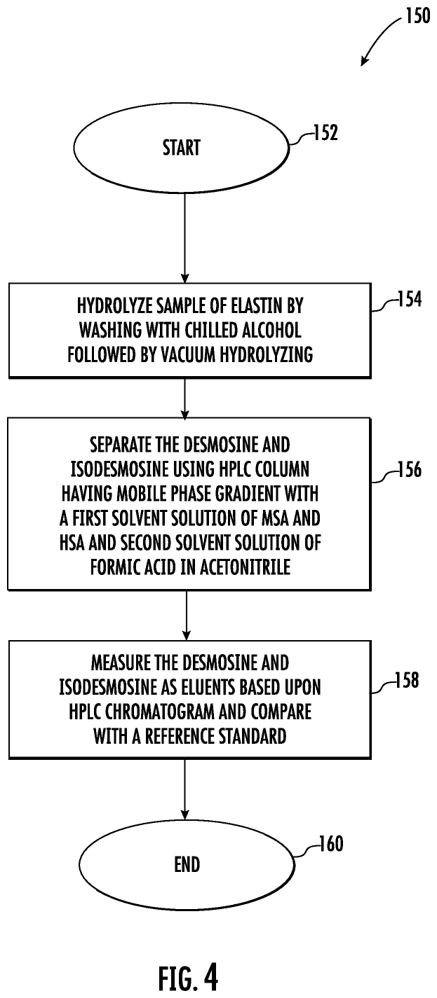

As shown in FIG. 4, a high-level process is illustrated for measuring desmosine and isodesmosine in elastin and shown generally at 150. The process starts (Block 152) and similar to the process of FIG. 3, the sample of elastin is hydrolyzed by washing with chilled alcohol followed by vacuum hydrolyzing (Block 154). The desmosine and isodesmosine are separated using a HPLC column having a mobile phase gradient with a first solvent solution of MSA and HSA, and a second solvent solution of formic acid and Acetonitrile (Block 156). The desmosine and isodesmosine are measured as eluents based upon a HPLC chromatogram and compared with a reference standard (Block 158). The process ends (Block 160).

There now follows an example of the process for not only isolating the desmosine and isodesmosine as eluents, but also measuring both such as in the process of FIG. 4.

A 50 mg sample of elastin powder, such as derived from Atlantic cod, is placed in a falcon tube and 1.8 mL of ice cold alcohol, and in this example, ethanol, are added. The sample is vortexed as a type of mixing for 30 seconds and let stand at about −20° Centigrade for about 10 minutes. In an example, the vortexing as mixing may be about 20 to 40 seconds and the sample may stand at about −30° to about −10° Centigrade for about 5 to 15 minutes. After this period, the sample is vortexed at about 13,000 rpm for about 10 minutes at about 4° Centigrade. This vortexing may occur for about 5 to 15 minutes at about 0° to 10° Centigrade, but no more than 10° Centigrade. The ethanol is removed and the washing is repeated once more. The washed elastin is retrieved from the tube using 2 mL of 6 N HCl in this example.

Some prior art techniques required washing with a mixture of 2:1 chloroform-methanol and then acetone. The washed sample is transferred to a 6 mL vacuum hydrolysis tube. The vacuum hydrolysis is carried out for about 24 hours at about 110° Centigrade, and in an example, the washed sample may be hydrolyzed at about 100° to about 120° C. entgrade for about 20 to about 28 hours. Prior art techniques require hydrolysis to be performed for 48 hours. After hydrolysis, the sample is dried out at about 60° Centigrade under a gentle stream of nitrogen (N₂), and in an example, at about 50° to about 70° Centigrade.

The dried sample is resuspended in 500 μL of 10% methanol and a 50 μL aliquot is diluted (1:1 v/v) with HPLC water for HPLC analysis. The methanol may vary from between about 9% to 11%. Desmosine and isodesmosine are separated by HPLC in a Nucleosil C18 column (150×4.6 mm, 5 μm; Sigma-Aldrich, St. Louis, Missouri) at about 35° C., using a mobile phase gradient of a first solvent as 0.1 M MSA (methane sulfonic acid) and 6 mM HSA (sodium hexanesulfonate) having a pH of about 2.0 (A), and a second solvent as 0.2% formic acid in Acetonitrile (B) [0-5% B in 5 minutes; 5-12% B in 15 minutes; 12-5% B in 15 to 25 minutes]. The first solvent solution (A) may be about 0.098 M to about 0.102 M MSA and about 5.88 mM to about 6.12 mM HSA at about pH 1.90 to about pH 2.1. The second solvent solution (B) may be about 0.196% to about 0.204% formic acid in Acetonitrile, and the gradient occur at about 0-5% in about 4-6 minutes, 5-12% in about 12-18 minutes, and 12-5% in about 12 to about 28 minutes. Desmosine and isodesmosine absorbance is measured at λ=275 nm as eluents from the HPLC column based upon the HPLC chromatogram.

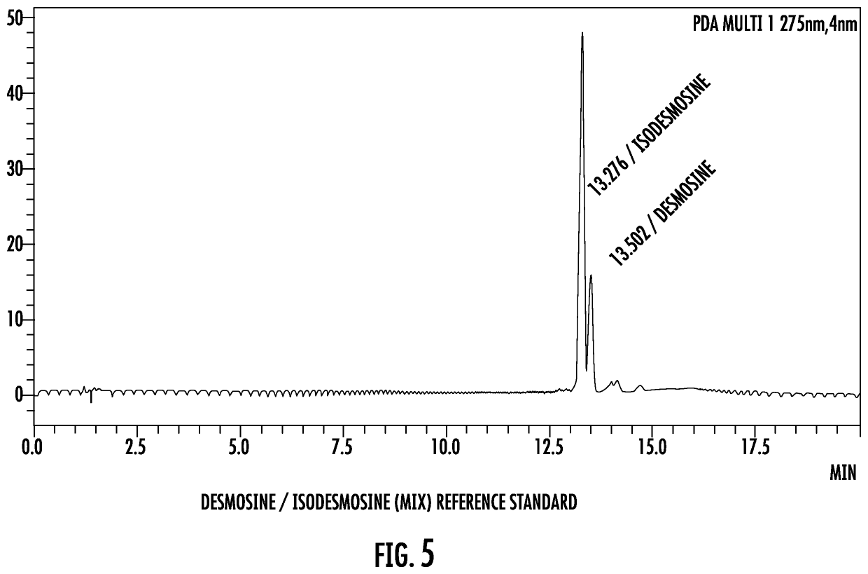

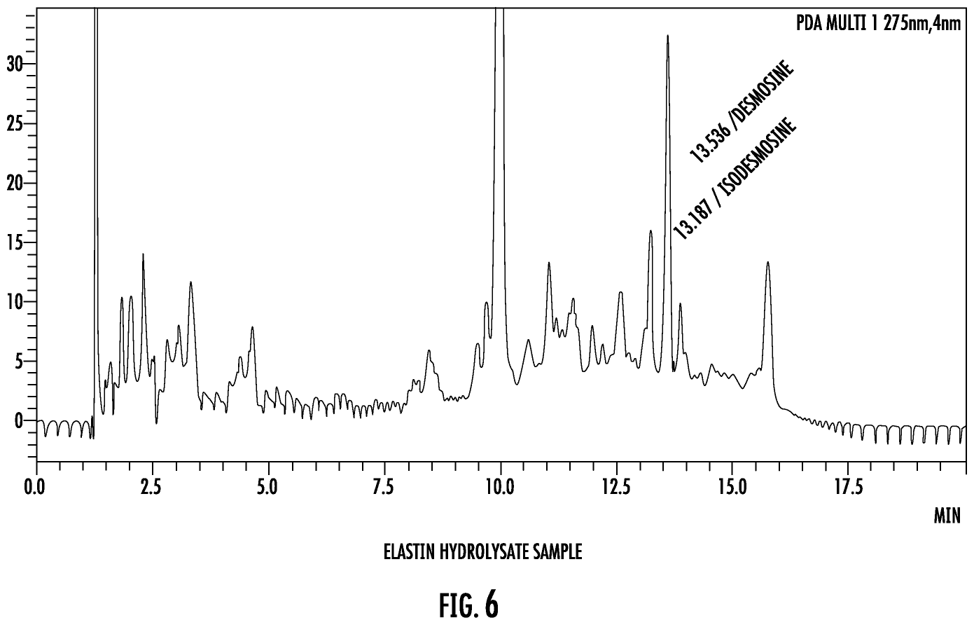

There now follows a more complete description of the data analysis and calculations. Desmosine and isodesmosine peaks in the chromatogram are identified based on their respective retention times, such as shown in the graph of FIG. 6, comparing them with a reference standard (FIG. 5) for each amino acid and then integrated in this example by LabSolutions (Shimadzu, Japan). Areas of peaks of each amino acid are recorded and used for calculations. The elastin hydrolysate sample of FIG. 6 is an example sample as ELASDERMAD manufactured by Nutraceuticals International Group LLC d/b/a Nutraceuticals Group.

The peak area for each amino acid is recorded. The amount of isodesmosine in μg/g in the sample is calculated as follows:

$$Isodesmosine , μg / g = \frac{{A}_{iso} - {b}_{iso}}{{m}_{iso}} \times \frac{V}{W}$$

where:
Aᵢₛₒ=peak area of isodesmosine in sample chromatogram;
bᵢₛₒ=y-intercept of calibration curve for isodesmosine;
mᵢₛₒ=slope of calibration curve for Isodesmosine;
V=volume of test solution=0.5 mL, W=sample weigh in g.

The amount of desmosine in μg/g in the sample is calculated as follows:

$$Desmosine , μg / g = \frac{{A}_{des} - {b}_{des}}{{m}_{des}} \times \frac{V}{W}$$

where:
A_{des}=peak area of desmosine in sample chromatogram;
b_{des}=y-intercept of calibration curve for desmosine;
m_{des}=slope of calibration curve for desmosine;
V=volume of test solution in mL, W=sample weigh in g.

The percentage of (w/w) is calculated from μg/g as follows:

$$\% ( w / w ) = \frac{μ g / g}{10000} .$$

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

1. A method of isolating desmosine and isodesmosine in elastin, comprising:
hydrolyzing a sample of elastin; and
separating the desmosine and isodesmosine in the hydrolyzed sample of elastin within a high-performance liquid chromatography (HPLC) column using a mobile phase gradient comprising a first solvent solution of MSA (methane sulfonic acid) and HSA (sodium hexanesulfonate) and a second solvent solution of formic acid in Acetonitrile.

2. The method of claim 1 wherein the HPLC column comprises a C18 HPLC column operating at about 30 to about 40 degrees Centigrade.

3. The method of claim 1 wherein the first solvent solution comprises about 0.098 M to about 0.102 M MSA and about mM to about 6.12 mM HSA at about pH 1.90 to about pH 2.1 and the second solvent solution comprises about 0.196% to about formic acid in Acetonitrile at about 0-5% in about 4 to about 6 minutes, 5-12% in about 12-18 minutes, and 12-5% in about 12 to about 28 minutes.

4. The method of claim 1 wherein the hydrolyzing comprises washing the sample of elastin in chilled alcohol at less than about 10 degrees Centigrade.

5. The method of claim 4 wherein the washing comprises mixing the sample of elastin with the chilled alcohol for about 20 to 40 seconds and letting stand at about −30 to about −10 degrees Centigrade for about 5 to 15 minutes, followed by mixing for about 5 to 15 minutes at about 0 to 10 degrees Centigrade.

6. The method of claim 4 comprising hydrolyzing the washed sample of elastin by vacuum hydrolysis with HCl.

7. The method of claim 4 comprising vacuum hydrolyzing the washed sample of elastin at about 100 to 120 degrees Centigrade for about 20 to 28 hours.

8. The method of claim 4 comprising drying the hydrolyzed sample of elastin using nitrogen gas at about 50 to about 70 degrees Centigrade.

9. A method of measuring desmosine and isodesmosine in elastin, comprising:
hydrolyzing a sample of elastin by washing the sample of elastin in chilled alcohol at less than 10 degrees Centigrade followed by vacuum hydrolyzing the washed sample of elastin within HCl;
separating the desmosine and isodesmosine in the hydrolysed sample of elastin within a high-performance liquid chromatography (HPLC) column using a mobile phase gradient comprising a first solvent solution of MSA (methane sulfonic acid) and HSA (sodium hexanesulfonate) and a second solvent solution of formic acid in Acetonitrile; and
measuring the amount of desmosine and isodesmosine as eluents from the HPLC column at about 275 nanometers based upon a HPLC chromatogram.

10. The method of claim 9 wherein the HPLC column comprises a C18 HPLC column operating at about 30 to about 40 degrees Centigrade.

11. The method of claim 9 wherein the first solvent solution comprises about 0.098 M to about 0.102 M MSA and about mM to about 6.12 mM HSA at about pH 1.90 to about pH 2.1 and the second solvent solution comprises about 0.196% to about formic acid in Acetonitrile at about 0-5% in about 4 to about 6 minutes, 5-12% in about 12-18 minutes, and 12-5% in about 12 to about 28 minutes.

12. The method of claim 9 wherein the washing comprises mixing the sample of elastin with the chilled alcohol for about 20 to 40 seconds and letting stand at about −30 to about −10 degrees Centigrade for about 5 to 15 minutes, followed by mixing for about 5 to 15 minutes at about 0 to 10 degrees Centigrade.

13. The method of claim 9 comprising vacuum hydrolyzing the washed sample of elastin at about 100 to 120 degrees Centigrade for about 20 to 28 hours.

14. The method of claim 9 comprising drying the hydrolyzed sample of elastin using nitrogen gas at about 50 to about 70 degrees Centigrade.

15. A method of measuring desmosine and isodesmosine in elastin, comprising:
hydrolyzing a sample of elastin by washing the sample of elastin in chilled alcohol at less than 10 degrees Centigrade followed by vacuum hydrolyzing the washed sample of elastin within HCl;
separating the desmosine and isodesmosine in the hydrolyzed sample of elastin within a high-performance liquid chromatography (HPLC) column using a mobile phase gradient comprising a first solvent solution comprising about 0.098 M to about 0.102 M MSA (methane sulfonic acid) and about 5.88 mM to about 6.12 mM HSA (sodium hexanesulfonate) at about pH 1.90 to about pH 2.1, and a second solvent solution comprising about 0.196% to about 0.204% formic acid in Acetonitrile at about 0-5% in about 4 to about 6 minutes, 5-12% in about 12-18 minutes, and 12-5% in about 12 to about 28 minutes; and
measuring the amount of desmosine and isodesmosine as eluents from the HPLC column at about 275 nanometers based upon a HPLC chromatogram.

16. The method of claim 15 wherein desomosine and isodesmosine peaks are identified in the chromatogram and compared with a reference standard.

17. The method of claim 15 wherein the HPLC column comprises a C18 HPLC column operating at about 30 to about 40 degrees Centigrade.

18. The method of claim 15 wherein the washing comprises mixing the sample of elastin with the chilled alcohol for about 20 to 40 seconds and letting stand at about −30 to about −10 degrees Centigrade for about 5 to 15 minutes, followed by mixing for about 5 to 15 minutes at about 0 to 10 degrees Centigrade.

19. The method of claim 15 comprising vacuum hydrolyzing the washed sample of elastin at about 100 to about 120 degrees Centigrade for about 20 to about 28 hours.

20. The method of claim 15 comprising drying the hydrolyzed sample of elastin using nitrogen gas at about 50 to about 70 degrees Centigrade.
